(19) United States
(12) Patent Application Publication
(10) Pub. No.: US 2013/0131566 A1
(43) Pub. Date: May 23, 2013

(54) CORE JACKET

(71) Applicant: Susan A. Bodansky, Potomac, MD (US)

(72) Inventor: Susan A. Bodansky, Potomac, MD (US)

(21) Appl. No.: 13/679,330

(22) Filed: Nov. 16, 2012

Related U.S. Application Data
(60) Provisional application No. 61/562,160, filed on Nov. 21, 2011.

(51) Int. Cl. A61F 5/37

(52) U.S. Cl. CPC A61F 5/37
USPC 602/13

(57) ABSTRACT

A core jacket including one or more weights and adapted to provide a compression force to a wearer of the jacket. The compression force may be created by one or more inflatable bladders disposed in the core jacket. The core jacket is adapted to provide a readily accessible tool to foster self-reliance in the wearer, increasing self-confidence and self/body awareness of the wearer. The core jacket may also be used as an educational device to assist the wearer in understanding and experiencing the body and mind connection.

### CROSS REFERENCES TO RELATED APPLICATIONS

This application claims the benefit of U.S. Provisional Patent Application Ser. No. 61/562,160, filed on Nov. 21, 2011, the contents of which are incorporated herein by reference in their entirety.

### FIELD

The present disclosure relates to devices and methods for therapeutic uses, and more particularly to wearable devices for therapeutic use.

### BACKGROUND

There are numerous individuals who have been or may be diagnosed with sensory integration dysfunction, attention deficit disorder, attention deficit hyperactivity disorder, Post-Traumatic Stress Disorder (PTSD) and other anxiety disorders, hyperactivity, autism, and other dysfunctions and disorders of the type. Those diagnosed with such a dysfunction or disorder may suffer from certain symptoms, for example, anxiety, an oversensitivity or under-sensitivity to touch, movement, sights, or sounds; a tendency to be easily distracted; social and/or emotional problems; an activity level that is abnormally high or low; a lacking in self-control and/or self-confidence; a difficulty to calm oneself down, and other symptoms.

Treatment of such disorders often involve treatments, such as occupational therapy, psychotherapy, prescription and non-prescription drugs, electrical stimulation, activity programs, therapeutic messages and touch, and other treatments. However, many of these treatments cannot be performed by the individuals on their own or in the comfort of their home.

### SUMMARY

The present application discloses wearable therapeutic devices and methods for therapeutic use, for example, treating an anxiety disorder and to facilitate the reduction of stress, anxiety, and tension. The wearable therapeutic devices may also assist in emotionally strengthening a user's body and core, simulate a feeling of being held or contained, and facilitate moving from a feeling of uncentered, dissociated or feeling of free-floating anxiety to a feeling of being grounded and a return of a strengthened core, independent functioning, and increased drive.

In an illustrative embodiment, the wearable therapeutic device is a core jacket having an external portion and an internal portion, and a closure mechanism on the jacket adapted to secure the jacket on a user. The jacket may include a first compartment disposed on the internal portion of the jacket that is adapted to receive a weight, and a second compartment disposed on the internal portion of the jacket that is adapted to receive a compression device. The compression device may be an inflatable bladder that provides a compression force to the user when the user is wearing the jacket. In another embodiment, the jacket may include strings or laces threaded through grommets adapted to be tightened to provide a compression force to the user when the user is wearing the jacket, for example, to simulate a feeling of being hugged.

The jacket may also include a detachable collar and a detachable hood disposed on a neck/head opening of the jacket. The hood can contain ear muffs that are adapted to provide another compression force to the user when the user is wearing the jacket, for example, to the ears of the user. An adjustable strap or straps may be disposed around the external portion of the jacket to maintain compression in an inward direction, and adapted to tighten the jacket around the user when the user is wearing the jacket.

### BRIEF DESCRIPTION OF THE DRAWINGS

Embodiments of devices and methods are illustrated in the figures of the accompanying drawings which are meant to be exemplary and not limiting, in which like references are intended to refer to like or corresponding parts, and in which:

FIG. 1 illustrates a front exterior view of an embodiment of a core jacket;

FIG. 2 illustrates an interior view of an embodiment of the core jacket;

FIG. 3 illustrates a back view of an embodiment of the core jacket;

FIG. 4 illustrates a view of another embodiment of a core jacket; and

FIG. 5 illustrates a flow diagram of a method of using the core jacket.

### DETAILED DESCRIPTION

Detailed embodiments of devices and methods are disclosed herein, however, it is to be understood that the disclosed embodiments are merely exemplary of the devices and methods, which may be embodied in various forms. Therefore, specific functional details disclosed herein are not to be interpreted as limiting, but merely as a basis for the claims and as a representative basis for teaching one skilled in the art to variously employ the present disclosure.

The disclosure relates to a core jacket and a method for therapeutic use of the core jacket. The core jacket promotes independence of a user in achieving “inner strength” and a feeling of well-being. It can be used as an educational device and/or a therapeutic tool during or in-between therapeutic sessions, or by the user himself/herself after being trained in such use or by following an instruction. The core jacket may facilitate the reduction of the user's level of anxiety and tension, and/or may help the user to instinctually be “in the present moment” (a mindful place) with increased focus on a task at hand. The core jacket may create a physical reminder of a strong core, and/or may also create an association to an activity where the user feels self-confident and/or a sense of well-being.

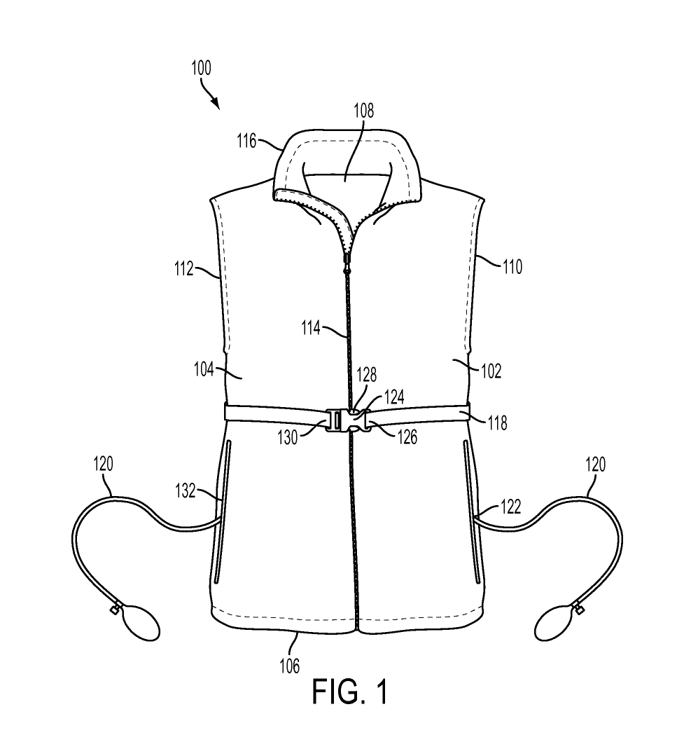

A front exterior view of the core jacket 100 according to an illustrative embodiment is described with reference to FIG. 1. As illustrated, the core jacket 100 is in the form of a vest having first front portion 102 and a second front portion 104, a bottom opening 106, a top or head and neck opening 108 opposite the bottom opening 106, a first arm opening 110, and a second arm opening 112 opposite the first arm opening 110. The bottom opening 106, the top or head and neck opening 108, the first arm opening 110, and the second arm opening 112 may be sized and shaped to fit a user or wearer of the core jacket 100.

The core jacket 100 also includes a front closure mechanism 114 extending from the bottom opening 106 to the top opening 108, a collar 116 disposed around the top opening 108, an adjustable strap 118 disposed around an exterior of the core jacket 100 between the bottom opening 106 and the top opening 108, one or more hoses 120 extending through one or more apertures 122 in the first front portion 102 and/or the second front portion 104, and one or more pockets 132.

As illustrated, the front closure mechanism 114 is a zipper that attaches and detaches the first front portion 102 and the second front portion 104 to and from one another, respectively. Although, the front closure mechanism 114 is illustrated as a zipper, the front closure mechanism 114 may be a series of buttons, snaps, hook-and-loop fasteners, and other closures of the type or a combination thereof.

The collar 116 is adapted to extend around a neck of the user. The collar 116 may also be attachable to and detachable from the core jacket 100. In other embodiments, the core jacket may alternatively or additionally include a hood disposed proximate to the top opening 108, and the hood may also be attachable to and detachable from the core jacket 100. The collar 116 and/or the hood may be attached to the core jacket 100 by a zipper, buttons, snaps, hook-and-loop fasteners, and other fasteners of the type or a combination thereof.

The adjustable strap 118 may further include adjustable closures or buckles. The adjustable closures or buckles may include a female fastener or clip 124 connected to a first end 126 of the strap 118 and a mating male fastener or clip 128 connected to a second end 130 of the strap 118. The adjustable closures or buckles may allow the user to tighten and loosen the length of the strap to securely fasten the core jacket 100 around the user, for example, around a lower back and abdomen of the user. In other embodiments, the adjustable strap 118 may be a belt or other fastening device that allows the user to tighten the core jacket 100 around the user.

The one or more pockets 132 are in or on the first front portion 102 and/or the second front portion 104, and may include a closure mechanism. The closure mechanism may be, for example, buttons, snaps, hook-and-loop fasteners, and other closures of the type or a combination thereof.

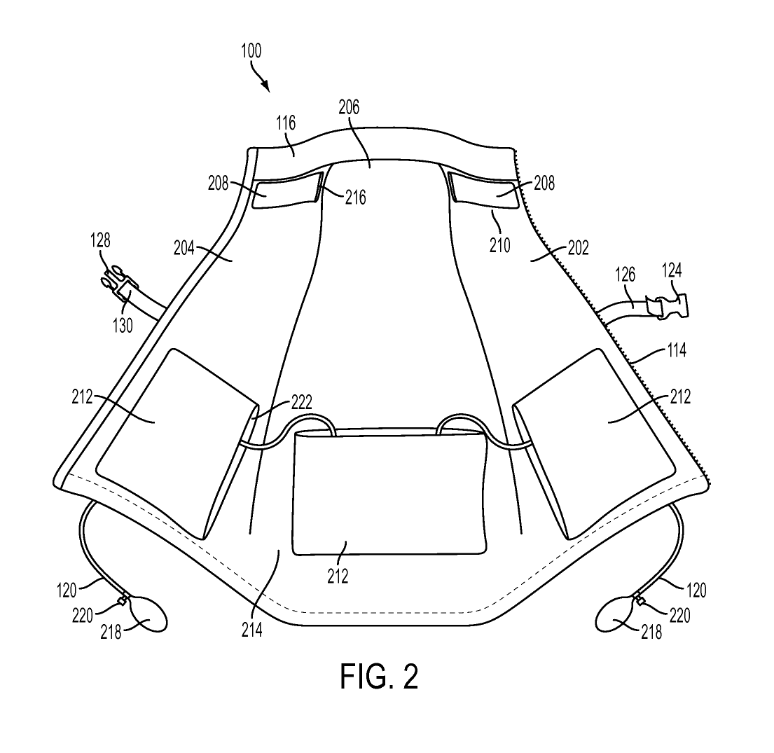

An interior view of the core jacket 100 according to an illustrative embodiment is described with reference to FIG. 2. As illustrated, the core jacket 100 includes a first internal portion 202 corresponding to an interior side of the first front portion 102, a second internal portion 204 corresponding to an interior side of the second front portion 104, and a rear internal portion 206. The core jacket 100 may also include one or more first compartments 208 disposed on at least one of the internal portions 202, 204, and 206 proximal to a shoulder area 210 of the core jacket 100, and one or more second compartments 212 disposed on at least one of the internal portions 202, 204, and 206 proximal to a waist or hip area 214 of the core jacket 100.

The one or more first compartments 208 may extend over the shoulders of a user wearing the core jacket 100. In one embodiment, a first compartment 208 is disposed to extend over a right shoulder of a user wearing the core jacket 100 and another first compartment 208 is disposed to extend over a left shoulder of the user wearing the core jacket 100. Weights may be disposed in the first compartments 208. The weights may be flexible weights adapted to form to a contour of the user's shoulders. The weights may also be shaped like a human hand to cause the user to feel like there is a hand positioned on the user's shoulders when the user is wearing the core jacket 100. The weights may include materials that cool, and/or retain, generate or emit heat or otherwise provide temperature elevation or reduction. The first compartments 208 may also include openings 216 having closure mechanisms, for example, zippers, buttons, snaps, hook-and-loop fasteners, such as Velcro, and other fasteners of the type or a combination thereof, allowing the insertion and removal of the weights disposed in the first compartments 208.

The one or more second compartments 212 may be disposed to extend around the waist, abdomen, and/or hips of the user wearing the core jacket 100. In one embodiment, there are one or more bladders disposed in the one or more second compartments 212 adapted to fill with gas such as air, or fluid, to provide compression force to the waist, abdomen, and/or hips of the user wearing the core jacket 100. As illustrated, there are three second compartments 212 each including an air bladder. The bladders may be configured similar to blood pressure cuffs or other forms of bladders adapted to provide a compression force to a user wearing the core jacket 100.

In this illustrative embodiment the air bladders are coupled to the hoses 120 which extend from the bladders. The hoses 120 include inflation bulbs 218 and release valves 220. The inflation bulbs 218 are adapted to be compressed or squeezed by a user to inflate the bladders, The release valves 220 are adapted to open the bladders to the atmosphere to deflate the bladders. Similar to the first compartments 208, the second compartments 212 may include openings 222 having closure mechanisms allowing the insertion and removal of the bladders disposed in the second compartments 212.

Although there are three second compartments 212 illustrated, each including a bladder, there may be a single second compartment 212 extending around the core jacket 100 including a single bladder. The bladder(s) may also include a manometer or other pressure measuring device as known in the art. The bladder(s) allow the user to create and self-regulate the amount of compressive force applied to the user wearing the core jacket 100. Additionally, the adjustable strap 118, or additional straps, may be positioned around the area where the compression will occur to further facilitate inward forces and allow the user to create and self-regulate the compressive force(s).

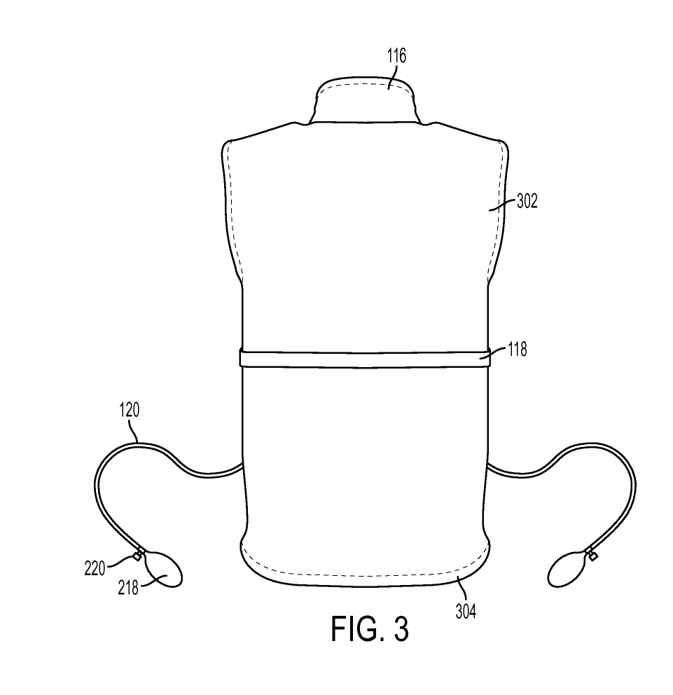

A rear external view of the core jacket 100 according to an illustrative embodiment is described with reference to FIG. 3. As illustrated, the core jacket 100 includes a rear portion 302, and a drawstring integrated into a bottom portion 304 of the core jacket 100 that extends around the bottom opening 106. The adjustable strap 118 may be attached, for example, by sewing, to the rear portion 302, and may be attached to the first front portion 102 and/or the second front portion 104.

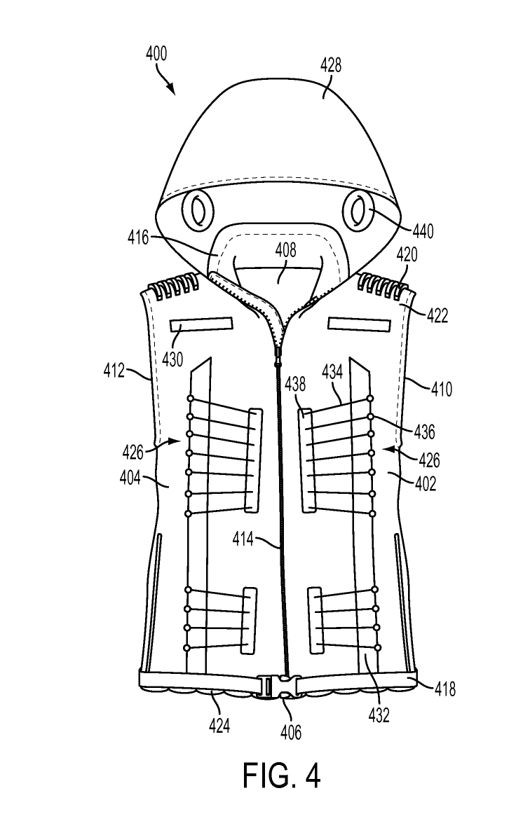

Another embodiment of the core jacket is described with reference to FIG. 4. In this embodiment, the core jacket 400 is similar to the core jacket 100 described above, and may include one or more alternative or additional features. Similar to the core jacket 100, the core jacket 400 may include a first front portion 402 and a second front portion 404, a bottom opening 406, a top or head and neck opening 408 opposite the bottom opening 406, a first arm opening 410, and a second arm opening 412 opposite the first arm opening 410. The core jacket 400 may also include a front closure mechanism 414 extending from the bottom opening 406 to the top opening 408, a collar 416 disposed around the top opening 408, and an adjustable strap 418. Similar to the core jacket 100, the core jacket 400 may include one or more bladders adapted to create a compression force simultaneously, or selectively, around the chest, shoulders, abdomen, waist, and/or hip area of a user wearing the core jacket 400. The adjustable strap 418 may be disposed around the core jacket 400 in an area corresponding to where the compression will occur.

As illustrated in FIG. 4, The core jacket 400 may include one or more first compartments 420 on shoulder portions 422 of the core jacket 400, one or more bottom compartments 424 disposed around the bottom opening 406 of the core jacket 400, a first and a second compression mechanism 426 disposed on the first and second front portions 402 and 404, respectively, and a hood 428. The jacket may include a scent portion 430 as an aromatherapy device, disposed on one or more of the first and second front portions 402 and 404.

The one or more first compartments 420 may be on an external surface of the core jacket 400 or an internal surface of the core jacket 400. The one or more first compartments 420 may also include one or more weights disposed in and removable from the first compartments 420. As illustrated there are five (5) first compartments 420 on a left shoulder portion and five (5) first compartments 420 on a right shoulder portion of the core jacket 400.

The one or more bottom compartments 424 may be on an external surface of the core jacket 400 or an internal surface of the core jacket 400, and may include one or more weights disposed in and removable from the bottom compartments 424. These bottom compartments 424 may be along the entire bottom of the vest and provide the user the ability to select where or if the user wishes to place weights.

The first and second compression mechanisms 426 may each include respective inflation or filling means. In this embodiment the jacket may include elastic portions 432, strings 434, grommets 436, and optionally hook-and-loop fasteners 438. The elastic portions 432 may be disposed on the respective first and second front portions 402 and 404 of the core jacket 400. The grommets 436 may be eyelets and may be disposed on the respective first and second front portions 402 and 404 along an edge of the elastic portions 432. The hook-and-loop fasteners 438 may also be disposed on the respective first and second front portions 402 and 404 on an opposite side of the respective elastic portions 432 as the respective grommets 436. The strings 434 are threaded through the grommets 436 and extend over the respective elastic portions 432 toward the closure mechanism 414 to the hook-and-loop fasteners 438. The strings 434 may be tightened or loosened by pulling on the strings to provide a desired compression force to the user of the core jacket 400.

The hood 428 is disposed proximate to the top opening 408, and may also be attachable to and detachable from the core jacket 400. The hood 428 may also include ear muffs 440 disposed in the hood 428. The ear muffs 440 may be adapted to provide a compression force, controllable by the user, to the ears of the user wearing the core jacket 400.

The scent portion 430 may be disposed on the core jacket 400 proximate to the top opening 408 and may include a “scratch and sniff” component. In another embodiment, the scent portion 430 may be a scent compartment adapted to receive a scent component. The scent components may provide different scents, for example, but not limited to cinnamon, mint, and other scents that may enhance or promote relaxation while using the jacket.

The core jacket described may also include a heating means for providing the ability of the core jacket to be heated, for example, similar to an electric blanket, either through the use of a battery or plug in/external power source. The heating means may also be one or more heating packs or any other device that may be placed in one or more of the first and/or second compartments in addition to or instead of the weights and/or the bladder. The heating packs are adapted to generate heat to the core jacket, and optionally to the collar and hood as well. The core jacket may include a cooling means for providing the ability of the core jacket to be cooled, for example, including but not limited to, electronic devices such as Peltier devices, ventilation and/or spaces for ice packs, which may optionally be incorporated into the collar and hood as well. Similar to the heating packs, the ice packs may be placed in one or more of the first and/or second compartments in addition to or instead of the weights and/or the bladder. Alternatively, the core jacket may include additional pockets or compartments for receiving the heating and/or cooling packs.

The core jacket may have an electronic compression device, similar to, but not limited to, an electronic controlled pressure cuff, for example, including a battery or plug in/external power source. The electronic compression device should allow for the amount of compression force to be controlled by the user. The core jacket may also include or be equipped with a computer chip adapted to measure one or more of, but not limited to, the compression force, temperature, amount of time worn, number of times worn, the weight of vest, and other parameters of the type related to the jacket and its use. The computer chip may also maintain data relating to the user, the jacket and/or the state of being of the user, and be adapted to be checked and/or monitored by medical personnel through wired or wireless electronic connection or communication with a computing device.

The weights described above may be one-half pound weights, one pound, or other sizes of weights that may be added to or removed from the core jacket according to the user's individual need and/or a doctor's advice. There may also be additional compartments for a greater amount of weight based on individual need and medical advice.

The core jacket(s) may be made out of a sturdy and soft material that is easy and comfortable for a user to put on and wear. The core jacket(s) may be constructed of one or more material layers. For example, the core jacket(s) may include an outer material layer, an inner material layer, and an insulation material dispose between the outer and inner material layers. In one embodiment, the core jacket(s) include a canvas material or strong material layer overlaid with a cotton or other softer fabric material layer.

The core jacket(s) may be created in different sizes and styles according to the needs of the user. For example, the core jacket(s) may be vests, long sleeve jackets, short sleeve jackets, and other types of apparel. The core jacket(s) may also be adapted to incorporate any number of the features described above in any number of configurations. Further, any of the methods and devices described herein for applying the weight, heating, cooling, and/or compression may be manual, computer assisted, battery powered, and/or powered by an external power source, or any combination.

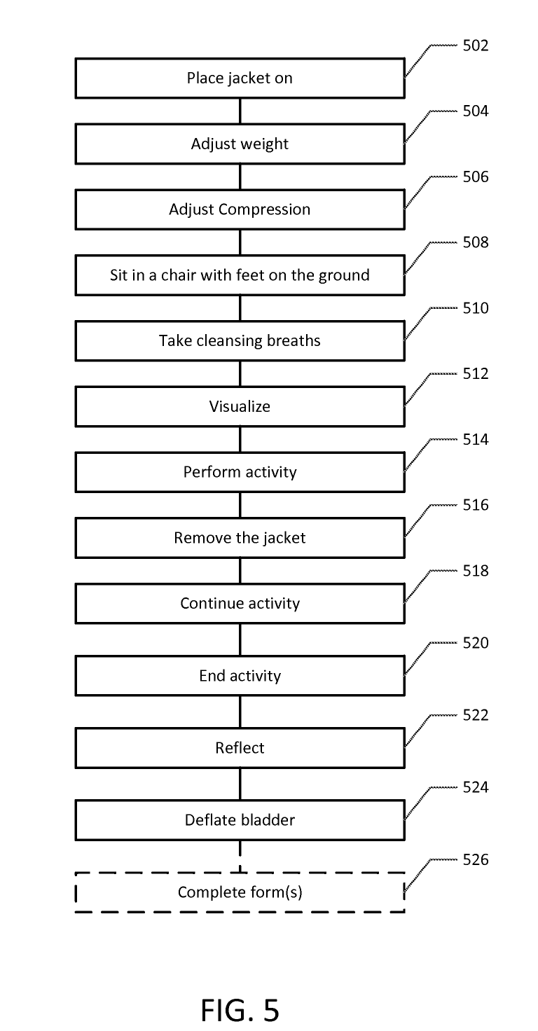

A flow diagram of a method of using the core jacket is described with reference to FIG. 5. In one example, a user may be an individual that is feeling “uncentered”, anxious, or disassociated, for example, as a result of a trauma, high functioning autism, sensory issues, or other cause. In another example, the core jacket may be used as a prevention tool or training tool for a user in the mind and body connection.

As illustrated in FIG. 5, initially, the user places the core jacket on, illustrated as block 502. The user adjusts the weight of the core jacket 504, for example, by adding or removing one or more weights from the core jacket. The user also adjusts the compression force of the core jacket 506, for example, by inflating or deflating the bladders, tightening the adjustable strap, and/or pulling on the strings. The weight and compression should be adjusted to an appropriate amount for the user, for example, based on medical advice from a doctor or therapist.

The user may then sit in a chair with his/her feet on the ground 508, and take a couple of deep or cleansing breaths, illustrated as block 510. It should be appreciated that if, at any time, the user feels uncomfortable during the usage of the core jacket, for example, lightheaded, dizzy, or short of breath, the user should immediately discontinue use of the core jacket and remove the core jacket.

The user may then use visualization techniques 512, to facilitate calming or “centering” of the user. The visualization may include imagery techniques, and may be adapted to the individual needs and preferences of the user. For example, the visualization may include imagining a beam of warm, yellow light coming down from head to toe; focusing on the user's breath going in and out; imagining the user's feet are on a piece of granite; and other techniques of the type. After a few minutes, or other amount of time, the user moves into performing an activity 514. The user should feel positive about the activity and the activity should engage the mind and optionally the body. The activity may include, for example, listening to music, singing or playing a musical instrument, drawing or creating a work of art, cooking, reading, or physical activity, for example, playing tennis, walking, or other activity. The individual should begin to feel a calming of the body and mind, a sense of “centering” or a feeling grounded or a feeling of being held or contained. During the activity, the user should focus the mind on the activity, and if the user's mind begins to wander, the user should gently return to involving the mind and body in the activity. The user should accept where the user is at with a non judgmental attitude of returning to the activity.

After a period of time, that may be an agreed upon time by the user in a consultation with a therapist, for example, about 15-30 minutes, the user removes the core jacket 516, and continues the activity, 518, for an additional time period. The user then ends the activity 520 and reflects 522, for example, by taking a moment to reflect on the imagery, activity and body sensations the user is feeling, and to take one or more deep cleansing breaths. When the user is finished, the user may deflate the core jacket, illustrated as block 524, and store the core jacket until the next use.

Based on the foregoing description, it can be seen that the core jacket provides a readily accessible tool to foster self-reliance in strengthening of the inner core of the user, and self/body awareness. The core jacket may be used as an educational device to assist the individual or user in understanding and experiencing the body and mind connection. The core jacket may also help improve the well-being of and provide relief to users who experience or suffer from anxiety, PTSD, sensory integration issues, hypersensitivity, dissociation, a feeling of being “uncentered”, attention deficit disorders, attention deficit hyperactivity disorders, autism, and other disorders, dysfunctions, and feelings of the type. Further, it should be appreciated that the core jacket may be used for other applications, such as, but not limited to, exercise or fitness apparel.

In an illustrative embodiment, the core jacket may include one or more forms, physical or electronic, as managed by an on-board computer chip, that the user would fill out (FIG. 5, 526) and send to the supplier of, manufacturer of, or doctor issuing the core jacket, for analyzing data relating to the jacket or it use or to assess the effectiveness and success of the core jacket. Each form would only include a number to identify the user for the study, and no other personal or identification information of the user. The form may request information from the user, for example, including, but not limited to, how often the user used the core jacket, when is it helpful, routine, and in what areas the core jacket has been found effective. The form may also request the user to provide feedback and indicate any problems or issues with the core jacket and any suggestions relating to the core jacket.

In one embodiment, there may be multiple forms. For example, a first form may be requested to be returned after one week of use by the user. A second form may be requested to be returned after one month of use by the user. A third form may be requested to be returned after six months of use by the user. A fourth form may be requested to be returned after one year of use by the user. The forms may be requested to be returned at different time intervals to evaluate the effectiveness and success of the core jacket.

It should be appreciated that although compartments described herein are disclosed as portions of the internal part of the core jacket, that such compartments may be alternatively implemented for holding weights and/or compression means, such as by configuring such compartments in external portions of the jacket.

Further, although the disclosure hereinbefore describes and illustrates specific numbers of compartments, bladders and/or straps, it should be appreciated that other embodiments may have different numbers or locations of compartments, bladders and/or straps for applying compression to a user according to need.

Although the devices and methods have been described and illustrated in connection with certain embodiments, many variations and modifications will be evident to those skilled in the art and may be made without departing from the spirit and scope of the disclosure. The discourse is thus not to be limited to the precise details of methodology or construction set forth above as such variations and modification are intended to be included within the scope of the disclosure. Moreover, unless specifically stated any use of the terms first, second, etc. do not denote any order or importance, but rather the terms first, second, etc. are merely used to distinguish one element from another.

What is claimed is:

1. A wearable therapeutic device, comprising:
a jacket having an external portion and an internal portion;
a closure mechanism on said jacket adapted to secure said jacket on a user,
a first compartment disposed on said internal portion of said jacket and adapted to receive a weight;
a bladder adapted to provide a compression force to said user when said user is wearing said jacket; and
a second compartment disposed on said internal portion of said jacket and adapted to receive said bladder.

2. The wearable therapeutic device as claimed in claim 1, wherein said bladder is an inflatable bladder.

3. The wearable therapeutic device as claimed in claim 2, wherein said inflatable bladder is electronically controlled for inflation.

4. The wearable therapeutic device as claimed in claim 1, further comprising a detachable collar disposed around a neck portion of said jacket.

5. The wearable therapeutic device as claimed in claim 1, further comprising a detachable hood disposed proximate a neck portion of said jacket.

6. The wearable therapeutic device as claimed in claim 5, wherein said hood contains ear muffs adapted to provide a compression force to said user when said user is wearing said jacket.

7. The wearable therapeutic device as claimed in claim 1, further comprising an adjustable strap disposed around said external portion of said jacket and adapted to tighten said jacket around said user when said user is wearing said jacket.

8. A wearable therapeutic device, comprising:
a jacket having a first portion and a second portion;
a first compartment disposed on said jacket and adapted to receive a weight; and
a compression mechanism disposed in one of said first or second portion of said jacket and adapted to provide a compression force to said user when said user is wearing said jacket.

9. The wearable therapeutic device as claimed in claim 8, wherein said compression mechanism includes grommets and strings threaded through said grommets.

10. The wearable therapeutic device as claimed in claim 8, further comprising a scent compartment disposed on said jacket proximate a neck portion of said jacket and adapted to receive a scented component.

11. The wearable therapeutic device as claimed in claim 8, wherein said first compartment is disposed on an internal portion of said jacket and around a waist portion of said jacket.

12. The wearable therapeutic device as claimed in claim 8, further comprising a detachable hood disposed proximate a neck portion of said jacket.

13. The wearable therapeutic device as claimed in claim 12, wherein said hood contains ear muffs adapted to provide a compression force to said user when said user is wearing said jacket.

14. The wearable therapeutic device as claimed in claim 8, further comprising an adjustable strap disposed around said external portion of said jacket proximate a waist portion of said jacket.

15. The wearable therapeutic device as claimed in claim 8, further comprising an inflatable bladder disposed on an internal portion of said jacket and adapted to provide a compression force to said user when said user is wearing said jacket.
